(19) United States
(12) Patent Application Publication
(10) Pub. No.: US 2006/0140896 A1
(43) Pub. Date: Jun. 29, 2006

(54) Cosmetic compositions comprising at least one silicone copolymer in a aqueous emulsion and at least one thickener, and uses thereof

(75) Inventors: Sandrine Decoster, Saint Gratien (FR); Veronique Douin, Paris (FR); Virginie Bailly, Clichy (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/288,409

(22) Filed: Nov. 29, 2005

Related U.S. Application Data
(63) Continuation of application No. 09/692,716, filed on Oct. 20, 2000, now abandoned.

(30) Foreign Application Priority Data
Oct. 20, 1999 (FR) 99 13099

(51) Int. Cl. A61K 8/89

(52) U.S. Cl. 424/70.12

(57) ABSTRACT

Cosmetic compositions comprising, in a cosmetically acceptable medium, at least one aqueous emulsion of at least one silicone copolymer with a dynamic viscosity ranging from 1×10⁶ to 100×10⁶ cP and at least one non-cellulose thickener. This combination can give cosmetic properties, such as at least one of smoothness, lightness, and softness, without the phenomenon of regreasing keratin fibers. These compositions can be used for washing and/or conditioning a keratin material, such as the hair or the skin.

The present invention relates to novel cosmetic compositions comprising, in a cosmetically acceptable medium, at least one non-cellulose thickener and at least one aqueous emulsion of at least one silicone copolymer defined below, with a dynamic viscosity ranging from 1×10⁶ to 100×10⁶ cP.

It is well known that hair that has been sensitized (i.e. damaged and/or embrittled) to varying degrees under the action of atmospheric agents or mechanical or chemical treatments, such as dyes, bleaches and/or permanent-waving, can be often difficult to disentangle and to style, and may lack softness.

It has already been recommended to use conditioners, in particular cationic polymers or silicones, in compositions for washing or caring for keratin materials such as the hair, in order to facilitate the disentangling of the hair and to give it softness and suppleness. However, the cosmetic advantages mentioned above can be accompanied, on dried hair, by certain cosmetic effects considered undesirable, i.e., lankness of the hairstyle (lack of lightness of the hair) and lack of smoothness (hair not uniform from the root to the tip).

In addition, the use of cationic polymers for this purpose may have various drawbacks. On account of their high affinity for the hair, some of these polymers can become deposited thereon to a large extent during repeated use, and may lead to adverse effects such as an unpleasant, laden feel, stiffening of the hair and interfiber adhesion which may affect styling. These drawbacks may be more accentuated in the case of fine hair, which lacks liveliness and body.

In summary, it is found that the current cosmetic compositions comprising silicones are not always entirely satisfactory.

The inventors have now discovered that the combination of an aqueous emulsion of at least one silicone copolymer defined below, with a dynamic viscosity ranging from 1×10⁶ to 100×10⁶ cP, with at least one non-cellulose thickener makes it possible to overcome at least one of these drawbacks.

Thus, after considerable research conducted in this matter, the inventors have found that by introducing an emulsion of a particular silicone copolymer into compositions, such as hair compositions, containing at least one non-cellulose thickener, it is possible to limit, or even eliminate, the at least one of the problems generally associated with the use of such compositions, i.e., for example, the lankness (charged feel following repeated applications) and the lack of smoothness and softness of the hair, while at the same time retaining at least one of the other advantageous cosmetic properties which are associated with conditioner-based compositions.

This combination can give cosmetic properties, such as at least one of smoothness, lightness, and softness, without the phenomenon of regressing keratin fibers.

Moreover, when applied to the skin, for example in the form of a bubble bath or shower gel, the compositions of the invention can provide an improvement in the softness of the skin.

Thus, according to the present invention, cosmetic compositions are now proposed comprising, in a cosmetically acceptable medium, at least one aqueous emulsion of at least one silicone copolymer defined below, wherein said copolymer has a dynamic viscosity ranging from 1×10⁶ to 100×10⁶ cP, and at least one non-cellulose thickener.

Another subject of the invention relates to the use of at least one aqueous emulsion of at least one silicone copolymer defined below, with a dynamic viscosity ranging from 1×10⁶ to 100×10⁶ cP, in, or for the manufacture of, a cosmetic composition comprising at least one non-cellulose thickener.

The various subjects of the invention will now be described in detail. All the meanings and definitions of the compounds used in the present invention given below are valid for all the subjects of the invention.

The silicone copolymer generally has a dynamic viscosity, measured at a temperature of about 25° C. and at a shear rate of 0.01 Hz for a stress of 1500 Pa, ranging from 1×10⁶ to 100×10⁶ cP, such as ranging from 5×10⁶ cP to 30×10⁶ cP.

All the dynamic viscosity measurements given in the present patent application were taken at a temperature of about 25° C., on a Carri-Medium CSL2-500 machine.

The at least one silicone copolymer present in the composition according to the invention is in the form of an aqueous emulsion.

The expression “aqueous emulsion” means an emulsion of oil-in-water type in which the at least one silicone copolymer is dispersed, such as in the form of particles or droplets, in the aqueous phase forming the continuous phase of the emulsion. This emulsion can be stabilized with a common emulsifying system.

This silicone emulsion can have a silicone droplet or particle size ranging from 10 nm to 50 μm, such as from 0.3 μm to 20 μm. The particle size is measured by laser granulometry.

The emulsifying system comprises at least one surfactant commonly used in silicone emulsions. These surfactants may be nonionic, cationic, anionic or amphoteric, or mixtures thereof, such as those described below.

The emulsifying system represents, for example, from 0.5% to 10% by weight relative to the total weight of the emulsion.

The at least one silicone copolymer results from the addition reaction, in the presence of a catalyst, of at least:
(a) one polysiloxane of formula (I): 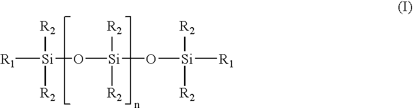
   in which:
   R₁, which may be identical or different, are independently chosen from groups that can react by chain addition reaction such as, for example, a hydrogen atom or aliphatic groups comprising an ethylenic unsaturation, such as vinyl, allyl and hexenyl groups;
   R₂ in formula (I), which may be identical or different, are independently chosen from hydroxyl, alkyl, alkenyl, cycloalkyl, aryl, and alkylaryl groups, and can optionally further comprise functional groups chosen from ethers, amines, carboxyls, hydroxyls, thiols, esters, sulfonates and sulfates; wherein:
      the alkyl groups comprise, for example, 1 to 20 carbon atoms; the alkenyl groups comprise, for example, from 2 to 10 carbon atoms; the cycloalkyl groups comprise, for example, 5 or 6 carbon atoms; the aryl groups comprise, for example, phenyl groups; and the alkylaryl groups comprise, for example, from 7 to 20 carbon atoms;
      In one embodiment, R₂ is chosen from methyl.
   n is an integer wherein the polysiloxane of formula (I) has a kinematic viscosity ranging from 1 to 1×10⁶ mm²/s, for example, n may range from 5 to 5000; and
(b) at least one silicone compound comprising at least one and not more than two groups capable of reacting with the groups R₁ of the polysiloxane (a), wherein:
   at least one of the compounds of type (a) and (b) comprises an aliphatic group, such as a C₂-C₆ aliphatic group, comprising an ethylenic unsaturation.

The compounds of type (b) can be another polysiloxane of type (a) in which at least one and not more than two groups R₁ of the polysiloxane (b) can react with the groups R₁ of the polysiloxane (a).

In one embodiment, the at least one silicone copolymer is obtained by addition reaction, in the presence of a hydrosilylation catalyst (for example a platinum catalyst), of at least:
(a) one α,ω-divinylpolydimethylsiloxane, and
(b) one α,ω-dihydrogenopolydimethylsiloxane.

The kinematic viscosity is measured, for example, at 25° C. according to ASTM standard 445 Appendix C.

The at least one silicone copolymer according to the invention is essentially non-crosslinked, i.e., not crosslinked to an extent sufficient to be referred to as a crosslinked copolymer.

The synthesis of these silicone emulsions is described for example in patent application EP-A-874 017, the disclosure of which is incorporated by reference herein.

Such emulsions are sold for example under the name DC2-1997 Cationic Emulsion by the company Dow Corning. This emulsion comprises an α,ω-divinyldimethicone/α,ω-dihydrogenodimethicone copolymer with a dynamic viscosity of about 15×10⁶ cP, an emulsifier of cationic type such as cetyltrimethylammonium chloride, a stabilizer such as hydroxyethylcellulose, and water.

The at least one silicone copolymer can be present in a representative amount ranging from 0.05% to 10% by weight relative to the total weight of the composition, such as from 0.1% to 5% by weight relative to the total weight of the composition.

The aqueous emulsion of the at least one silicone copolymer can be present in a representative amount ranging from 0.5% to 15% by weight relative to the total weight of the composition.

The non-cellulose thickeners according to the invention may be chosen from natural thickeners and synthetic origin thickeners.

Representative natural thickeners which are suitable for the invention include xanthan gum, scleroglucan gum, gellan gum, rhamsan gum, alginates, maltodextrin, starch and its derivatives, karaya gum, carob flour and guar gums. It is also possible to use these compounds after chemical modification, such as, for example, hydroxypropyl guars.

The purely synthetic thickeners according to the invention can be chosen from acrylic and methacrylic acid polymers and copolymers, such as acrylic acid/ethyl acrylate copolymers and carboxyvinyl polymers. Examples of such polymers and copolymers are the “Carbomer” (International Cosmetic Ingredient Dictionary and Handbook. 1997, hereafter, “CTFA”) products sold by the company Goodrich under the names Carbopol (Carbopol 980, 981, 954, 2984, 5984) and Synthalen, the polyglyceryl methacrylate sold by the company Guardian under the name Lubragel, and the polyglyceryl acrylate sold under the name Hispagel by the company Hispano Chimica.

Polyethylene glycols (PEG) and derivatives thereof may also be used as thickeners.

Thickening polyacrylamides may also be used for example as thickeners. These agents may be chosen for example from:
crosslinked 2-acrylamido-2-methylpropanesulfonic homopolymers,
optionally crosslinked copolymers of acrylamide and of ammonium acrylate,
optionally crosslinked copolymers of acrylamide (and of methacrylamide) and of methacryloyloxyethyltrimethylammonium chloride,
optionally crosslinked, partially and totally neutralized copolymers of acrylamide and of 2-acrylamido-2-methylpropanesulfonic acid.

Representative crosslinked copolymers of acrylamide/ammonium acrylate include acrylamide/ammonium acrylate copolymers (5/95 by weight) crosslinked with a crosslinking agent comprising polyolefinic unsaturation, such as divinylbenzene, tetraallyloxyethane, methylenebisacrylamide, diallyl ether, polyallylpolyglyceryl ethers and allyl ethers of alcohols of the sugar series, such as erythritol, pentaerythritol, arabitol, mannitol, sorbitol and glucose.

Similar copolymers are described and prepared in French patent FR-2 416 723 and U.S. Pat. Nos. 2,798,053 and 2,923,692, the disclosures of which are incorporated by reference herein.

This type of crosslinked copolymer is used for example in the form of a water-in-oil emulsion comprising about 30% by weight of said copolymer, 25% by weight of liquid paraffin, 4% by weight of a mixture of sorbitan stearate and of a hydrophilic ethoxylated derivative, and 41% by weight of water. Such an emulsion is sold under the name “Bozepol C” by the company Hoechst.

The copolymers of acrylamide and of 2-acrylamido-2-methylpropanesulfonic acid, that are used in accordance with the present invention, are copolymers crosslinked with a compound comprising polyolefinic unsaturation, such as those mentioned above, and partially or totally neutralized with a neutralizing agent chosen from, for example, sodium hydroxide, potassium hydroxide, aqueous ammonia and amines such as amines chosen from triethanolamine and monoethanolamine.

The copolymers of acrylamide and of 2-acrylamido-2-methylpropanesulfonic acid can be prepared by copolymerizing acrylamide and sodium 2-acrylamido-2-methylpropanesulfonate by way of a radical-mediated route by means of initiators such as azobisisobutyronitrile and by precipitation in an alcohol such as tert-butanol.

In one embodiment of the invention, copolymers obtained by copolymerization of 70 mol % to 55 mol % of acrylamide and of 30 mol % to 45 mol % of sodium 2-acrylamido-2-methylpropanesulfonate can be used. The crosslinking agent can be used, for example, in an amount ranging from 10⁻⁴ to 4×10⁻⁴ mol per mole of the mixture of monomers.

These specific copolymers can be incorporated into the compositions of the invention in the form of water-in-oil emulsions comprising from 35% to 40% by weight of this copolymer, from 15% to 25% by weight of a mixture of C₁₂-C₁₃ isoparaffinic hydrocarbons, from 3% to 8% by weight of polyethylene glycol lauryl ether comprising 7 mol of ethylene oxide, and water. Such an emulsion is sold under the name “Sepigel 305” by the company SEPPIC.

The crosslinked copolymer of acrylamide and of methacryloyloxyethyltrimethylammonium chloride, used according to the invention, can be chosen from copolymers obtained by copolymerization of acrylamide and of dimethylaminoethyl methacrylate quaternized with methyl chloride, followed by crosslinking with a compound comprising olefinic unsaturation, for example, methylenebisacrylamide.

In one embodiment, a crosslinked acrylamide/methacryloyloxy-ethyltrimethylammonium chloride copolymer (about 50/50 by weight) in the form of a dispersion comprising 50% by weight of said copolymer in mineral oil can be used. This dispersion is sold, for example, under the name “Salcare SC92” by the company Allied Colloids.

A crosslinked methacryloyloxyethyltrimethylammonium chloride homopolymer, optionally in the form of an inverse dispersion, may be used. These dispersions are sold, for example, under the names “Salcare SC95” and “Salcare SC96” by the company Allied Colloids.

The non-crosslinked copolymers of methacrylamide and of methacryloyloxyethyltrimethylammonium chloride are, for example, the products sold under the trade names Rohagit KF 400 and KF720 by the company Rohm & Haas.

The non-cellulose thickening polymers in cosmetic compositions in accordance with the present invention can have, in solution or in dispersion, at a concentration of 1% active material in water, a dynamic viscosity, measured using a Rheomat RM 180 rheometer at 25° C., of greater than 0.1 ps, and even greater than 0.2 cp, at a shear rate of 200 s⁻¹.

The at least one non-cellulose thickener can be present in an amount ranging, for example, from 0.001% to 20% by weight, such as from 0.01% to 10% by weight and further such as from 0.1% to 3% by weight, relative to the total weight of the final composition.

The compositions of the invention can also comprise at least one surfactant chosen from anionic, amphoteric and nonionic surfactants, which is generally present in an amount ranging from approximately 0.1% to 60% by weight relative to the total weight of the composition, such as from 3% to 40% and further such as from 5% to 30%.

The at least one surfactant chosen from anionic, amphoteric and nonionic surfactants, which are suitable for carrying out the present invention are, for example, the following:

(i) Anionic Surfactant(s):

In the context of the present invention, their nature is not of critical importance.

Representative anionic surfactants include salts (for example alkaline salts, such as sodium salts, ammonium salts, amine salts, amino alcohol salts and magnesium salts) of the following compounds: alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfdsuccinates; alkyl sulfosuccinamates; alkyl sulfoacetates; alkyl ether phosphates; acyl sarcosinates; acyl isethionates and N-acyltaurates. The alkyl and acyl radicals of all of these various compounds can for example comprise from 8 to 24 carbon atoms, and the aryl radicals can for example be chosen from phenyl and benzyl groups.

For example, anionic surfactants can-be chosen from fatty acid salts such as the salts of oleic, ricinoleic, palmitic and stearic acids, coconut oil acid and hydrogenated coconut oil acid and acyl lactylates in which the acyl radical comprises from 8 to 20 carbon atoms. At least one weakly anionic surfactant can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups.

As a further example, the anionic surfactant can be at least one salt chosen from alkyl sulfate salts and alkyl ether sulfate salts. (ii) Nonionic Surfactant(s):

Useful nonionic surfactants include compounds that are well known per se (see for example in this respect “Handbook of Surfactants” by M. R. Porter, published by Blackie & Son (Glasgow and London), 1991, pp. 116-178), the disclosure of which is incorporated by reference herein, and, in the context of the present invention, their nature is not a critical feature. Thus, nonionic surfactants can include polyethoxylated, polypropoxylated and polyglycerolated fatty acids, alkylphenols, α-diols and alcohols having a fatty aliphatic chain comprising, for example, 8 to 18 carbon atoms, it being possible for the number of ethylene oxide and propylene oxide groups to range for example from 2 to 50 and for the number of glycerol groups to range for example from 2 to 30. Mention may also be made of copolymers of ethylene oxide and of propylene oxide, condensates of ethylene oxide and of propylene oxide with fatty alcohols; polyethoxylated fatty amides for example comprising from 2 to 30 mol of ethylene oxide, polyglycerolated fatty amides comprising on average 1 to 5, such as from 1.5 to 4, glycerol groups; polyethoxylated fatty amines for example comprising from 2 to 30 mol of ethylene oxide; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides and N-acylaminopropylmorpholine oxides. It will be noted that the alkylpolyglycosides are nonionic surfactants that can be suitable in the context of the present invention.

(iii) Amghoteric Surfactant(s):

Representative amphoteric surfactants, whose nature is not a critical feature in the context of the present invention, can be chosen from aliphatic secondary and tertiary amine derivatives in which the aliphatic radical is chosen from linear and branched chain radicals comprising 8 to 22 carbon atoms and comprising at least one water-soluble anionic group (chosen for example from carboxylate, sulfonate, sulfate, phosphate and phosphonate); mention may also be made of (C₈-C₂₀)alkylbetaines, sulfobetaines, (C₈-C₂₀)alkylamido(C₁-C₆)alkylbetaines and (C₈-C₂₀)alkylamido(C₁-C₆)alkylsulfobetaines.

Representative amine derivatives include the products sold under the name Miranol, as described in U.S. Pat. Nos. 2,528,378 and 2,781,354, the disclosures of which are incorporated by reference herein, and having the structures:
R₂—CONHCH₂CH₂—N⁺(R₃)(R₄)(CH₂COO—) (2)
in which:
R₂ is chosen from alkyl radicals derived from an acid R₂—COOH present in hydrolysed coconut oil, heptyl, nonyl and undecyl radicals,
R₃ is chosen from β-hydroxyethyl groups, and
R₄ is chosen from carboxymethyl groups;
   and
   R₅—CONHCH₂CH₂—N(B)(C) (3)
   in which:
(B) is —CH₂CH₂OX′, with X′ chosen from a —CH₂CH₂—COOH group and a hydrogen atom,
(C) is —(CH₂)_{z}—Y′, with z=1 or 2, and with Y′ chosen from —COOH and —CH₂—CHOH—SO₃H radicals,
R₅ is chosen from alkyl radicals, such as (a) alkyl radicals of an acid R₅—COOH present in oils chosen from coconut oil and hydrolysed linseed oil, (b) alkyl radicals, such as C₇, C₉, C₁₁ and C₁₃ alkyl radicals, and (c) C₁₇ alkyl radicals and the iso forms, and unsaturated C₁₇ radicals.

Such representative compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloampho-dipropionate, lauroamphodipropionic acid, and cocoamphodipropionic acid.

By way of example, mention may be made of the cocoamphodiacetate sold under the trade name Miranol C2M Concentrate by the company Rhône-Poulenc.

In the compositions in accordance with the invention, at least two surfactants of different types can be used. Representative compositions include compositions comprising (a) more than one anionic surfactant, (b) at least one anionic surfactant and at least one amphoteric surfactant, and (c) at least one anionic surfactant and at least one nonionic surfactant. In one embodiment, the composition can comprise at least one anionic surfactant and at least one amphoteric surfactant.

The at least one anionic surfactant used for example, can be chosen from (C₁₂-C₁₄)alkyl sulfates of sodium, of triethanolamine and of ammonium; (C₁₂-C₁₄)alkyl ether sulfates of sodium, of triethanolamine and of ammonium, oxyethylenated with 2.2 mol of ethylene oxide; sodium cocoyl isethionate; and sodium (C₁₄-C₁₆)-α-olefin sulfonate, and used in combination with an amphoteric surfactant chosen from either:
amphoteric surfactants such as the amine derivatives known as disodium cocoamphodipropionate and sodium cocoamphopropionate, sold for example by the company Rhône-Poulenc under the trade name “Miranol C2M Conc®)” as an aqueous solution comprising 38% active material, and under the name Miranol C32; or
amphoteric surfactants of zwitterionic type, such as alkylbetaines, for example the cocobetaine sold under the name “Dehyton AB 30” as an aqueous solution comprising 32% AM by the company Henkel.

In one embodiment of the invention, the compositions can also comprise at least one cationic surfactant.

Representative at least one cationic surfactants can be chosen from salts of optionally polyoxyalkylenated primary, secondary and tertiary fatty amines; quaternary ammonium salts; imidazoline derivatives; and amine oxides of cationic nature.

The cationic surfactants may, for example, be chosen from:

A) quaternary ammonium salts of formula (IV) below: 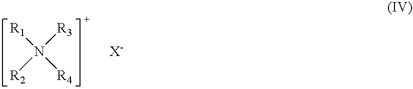
in which:
the radicals R₁, R₂, R₃, and R₄, which may be identical or different, are independently chosen from linear and branched aliphatic radicals comprising from 1 to 30 carbon atoms, and aromatic radicals, such as C₈-C₂₀ aromatic radicals (for example, aryl and alkylaryl), wherein the aliphatic radicals can comprise hetero atoms such as, oxygen, nitrogen, sulfur and halogens, and wherein the aliphatic radicals are chosen, for example, from alkyl, alkoxy, polyoxy(C₂-C₆)alkylene, alkylamide, (C₁₂-C₂₂)alkylamido(C₂-C₆)alkyl, (C₁₂-C₂₂)alkylacetate and hydroxyalkyl radicals, comprising from 1 to 30 carbon atoms;
X⁻ is an anion chosen from halides, phosphates, anions derived from organic acids, (C₂-C₆)alkyl sulfates, alkyl sulfonates, and alkylaryl sulfonates.

The compounds of formula (IV) can be chosen from, for example, (a) compounds comprising at least two fatty aliphatic radicals comprising from 8 to 30 carbon atoms, (b) compounds comprising at least one fatty aliphatic radical comprising from 17 to 30 carbon atoms, and (c) compounds comprising at least one aromatic radical.

B) Quaternary ammonium salts of imidazolinium, such as, for example, the salts of formula (V) below: 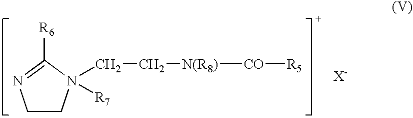
in which:
R₅ is chosen from alkenyl and alkyl radicals comprising from 8 to 30 carbon atoms, for example radicals derived from tallow fatty acid,
R₆ is chosen from a hydrogen atom, C₁-C₄ alkyl radicals, and alkenyl and alkyl radicals comprising from 8 to 30 carbon atoms,
R₇ is chosen from C₁-C₄ alkyl radicals,
R₈ is chosen from a hydrogen atom and C₁-C₄ alkyl radicals,
X⁻ is an anion chosen-from halides, phosphates, acetates, lactates, alkyl sulfates, alkyl sulfonates, and alkylaryl sulfonates.
For example, R₅ and R₆, which may be identical or different, are independently chosen from alkenyl and alkyl radicals comprising from 12 to 21 carbon atoms, for example, radicals derived from tallow fatty acid,
R₇ is methyl, and
R₈ is hydrogen.
Such products are, for example, (1) Quaternium-27 (International Cosmetic Ingredient Dictionary and Handbook, hereafter “CTFA”, 1997), i.e., “Rewoquat” W75, W75PG, and W90, and (2) Quaternium-83 (CTFA 1997), i.e., “Rewoquat” W75HPG, which are sold by the company Witco.

C) Diquaternary ammonium salts of formula (VI): 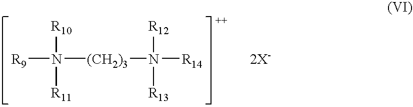
in which:
R₉ is chosen from aliphatic radicals comprising from 16 to 30 carbon atoms,
R₁₀, R₁₁, R₁₂, R₁₃ and R₁₄, which may be identical or different, are independently chosen from a hydrogen atom and alkyl radicals comprising from 1 to 4 carbon atoms, and
X⁻ is an anion chosen from halides, acetates, phosphates, nitrates and methyl sulfates.
For example, such diquaternary ammonium salts can comprise propane tallow diammonium dichloride.

D) Quaternary ammonium salts comprising at least one ester function. The quaternary ammonium salts comprising at least one ester function that can be used according to the invention are, for example, those of formula (VII) below: 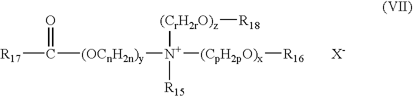
in which:
R₁₅ is chosen from C₁-C₆ alkyl radicals and C₁-C₆ hydroxyalkyl and C₁-C₆ dihydroxyalkyl radicals;
R₁₆ is chosen from:
   acyl groups of the following formula: 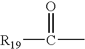
   wherein R₁₉ is defined below,
   linear and branched, saturated and unsaturated, C₁-C₂₂ hydrocarbon-based radicals, and
   a hydrogen atom;
R₁₈ is chosen from:
   acyl groups of the following formula: 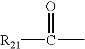
   wherein R₂₁ is defined below,
   linear and branched, saturated and unsaturated, C₁-C₆ hydrocarbon-based radicals, and
   a hydrogen atom;
   R₁₇, R₁₉ and R₂₁, which may be identical or different, are independently chosen from linear and branched, saturated and unsaturated, C₇-C₂₁ hydrocarbon-based radicals;
   n, p and r, which may be identical or different, are independently integers ranging from 2 to 6;
   y is an integer ranging from 1 to 10;
   x and z, which may be identical or different, are independently integers ranging from 0 to 10;
   X⁻ is chosen from simple and complex, organic and inorganic anions;
provided that the sum x+y+z is from 1 to 15, and that when x is 0, then R₁₆ is chosen from linear and branched, saturated and unsaturated, C₁-C₂₂ hydrocarbon-based radicals, and that when z is 0, then R₁₈ is chosen from linear and branched, saturated and unsaturated, C₁-C₆ hydrocarbon-based radicals.

In one embodiment, the R₁₅ alkyl radicals may be linear and branched and further, for example, linear.

For example, R₁₅ may be chosen from methyl, ethyl, hydroxyethyl and dihydroxypropyl radicals and further for example from methyl and ethyl radicals.

The sum x+y+z may for example range from 1 to 10.

When R₁₆ is chosen from linear and branched, saturated and unsaturated, C₁-C₂₂ hydrocarbon-based radicals, R₁₆ may be long and comprise from 12 to 22 carbon atoms, or short and comprise from 1 to 3 carbon atoms.

When R₁₈ is chosen from linear and branched, saturated and unsaturated, C₁-C₆ hydrocarbon-based radicals, R₁₈ may for example comprise from 1 to 3 carbon atoms.

R₁₇, R₁₉ and R₂₁, which may be identical or different, can, for example, be independently chosen from linear and branched, saturated and unsaturated C₁-C₂₁ hydrocarbon-based radicals, and for example from linear and branched, saturated and unsaturated, C₁₁-C₂₁, alkyl and alkenyl radicals.

x and z, which may be identical or different, can for example independently be chosen from 0 or 1.

y for example may be equal to 1.

n, p and r, which may be identical or different, can for example be independently chosen from 2 and 3 and in one embodiment equal to 2.

The anion for example can be chosen from halides (chloride, bromide, and iodide) and alkyl sulfates, such as methyl sulfate. However, methanesulfonate, phosphate, nitrate, tosylate, anions derived from organic acids, such as acetate and lactate, and any other anions compatible with the ammonium comprising an ester function, may be used.

As a further example, the anion X can be chosen from chloride and methyl sulfate.

Further examples of ammonium salts of formula (VII) are those in which:
R₁₅ is chosen from methyl and ethyl radicals,
x and y are equal to 1;
z is equal to 0 or 1;
n, p and r are equal to 2;
R₁₆ is chosen from:
   acyl radicals 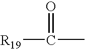
   wherein R₁₉ is defined below,
   methyl, ethyl and C₁₄-C₂₂ hydrocarbon-based radicals, and
   a hydrogen atom;
R₁₈ is chosen from:
   acyl radicals 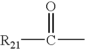
   wherein R₂₁, is defined below,
   a hydrogen atom;
R₁₇, R₁₉ and R₂₁, which may be identical or different, are independently chosen from linear and branched, saturated and unsaturated, C₁₃-C₁₇ hydrocarbon-based radicals, such as from linear and branched, saturated and unsaturated C₁₃-C₁₇ alkyl and alkenyl radicals.

The hydrocarbon-based radicals can for example be linear.

Representative compounds of formula (VII) are chosen from diacyloxyethyl-dimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (for example chloride and methyl sulfate). The acyl radicals can for example comprise from 14 to 18 carbon atoms and can for example be obtained from plant oils, such as palm oil and sunflower oil. When the compound comprises several acyl radicals, these radicals, which may be independently chosen, may independently be identical or different.

These products are obtained, for example, by direct esterification of compounds chosen from triethanolamine, triisopropanolamine, alkyldiethanolamines and alkyldiisopropanolamines, which are optionally oxyalkylenated, with fatty acids or with fatty acid mixtures of plant or animal origin, and by transesterification of the methyl esters thereof. This esterification is followed by a quaternization using an alkylating agent such as alkyl halides (such ad methyl and ethyl halides), dialkyl sulfates (for example dimethyl and diethyl sulfates), methyl methanesulfonate, methyl paratoluenesulfonate, glycol chlorohydrin and glycerol chlorohydrin.

Such compounds are sold, for example, under the names Dehyquart by the company Henkel, Stepanquat by the company Stepan, Noxamium by the company Ceca and Rewoquat WE 18 by the company Rewo-Witco.

It is also possible to use the ammonium salts comprising at least one ester function, described in patents U.S. Pat. Nos. 4,874,554 and U.S. Pat. Nos. 4,137,180, the disclosures of which are incorporated by reference herein.

Representative quaternary ammonium salts of formula (IV) include tetraalkylammonium chlorides such as, for example, dialkyldimethylammonium chlorides and alkyltrimethylammonium chlorides, in which the alkyl radical comprises from 12 to 22 carbon atoms, for example behenyltrimethylammonium chloride, distearyldimethylammonium chloride, cetyltrimethylammonium chloride, and benzyldimethylstearylammonium chloride, and, stearamidopropyldimethyl(myristyl acetate)ammonium chloride sold under the name “Cepharyl 70” by the company Van Dyk.

According to the invention, the at least one cationic surfactant can for example be present in an amount ranging from 0.1% to 20% by weight relative to the total weight of the final composition, such as from 0.1% to 10%, from 0.5% to 7%, and further such as from 1% to 5% by weight relative to the total weight of the final composition.

The composition of the invention can also comprise at least one additive chosen from fragrances, nacreous agents, preserving agents, silicone sunscreens, non-silicone sunscreens, vitamins, provitamins, cationic, amphoteric, anionic and nonionic polymers, proteins, protein hydrolysates, 18-methyleicosanoic acid, hydroxy acids, panthenol, volatile and non-volatile, cyclic and linear and crosslinked, modified and non-modified silicones, ceramides, pseudoceramides, plant, animal, mineral and synthetic oils and any other additive conventionally used in cosmetics which does not substantially adversely affect the properties of the compositions according to the invention.

Generally, these additives are present in the composition according to the invention in amounts, for example, ranging from 0 to 20% by weight relative to the total weight of the composition. The precise amount of each additive is readily determined by those skilled in the art depending on its nature and its function.

The compositions in accordance with the invention can also be used for washing or treating at least one keratin material chosen from hair, skin, eyelashes, eyebrows, nails, lips, scalp, and hair.

The compositions according to the invention can also be a detergent composition chosen from shampoos, shower gels, bubble baths and make-up-removing products. In this embodiment of the invention, the compositions comprise a washing base, which is generally aqueous.

At least one surfactant forms the washing base and can be chosen from anionic, amphoteric, nonionic and cationic surfactants, such as those defined above.

The quantity and quality of the washing base are sufficient to give the final composition at least one of the following qualities, satisfactory foaming power and satisfactory detergent power.

According to the invention, the washing base can be present for example in an amount ranging from 4% to 50% by weight, such as from 6% to 35% by weight and, even further such as from 8% to 25% by weight, relative to the total weight of the final composition.

Another subject of the invention is also a process for treating keratin materials such as the skin and the hair, comprising applying a cosmetic composition as defined above to the keratin materials and optionally rinsing it out with water.

Thus, this process according to the invention allows maintenance of the hairstyle and treatment of, care of, washing of or removal of make-up from the skin, the hair or any other keratin material.

The compositions of the invention can for example be in forms chosen from rinse-out conditioners and leave-in conditioners; permanent-waving, straightening, dyeing and bleaching compositions; rinse-out compositions to be applied before a procedure chosen from dyeing, bleaching, permanent-waving and straightening the hair; rinse-out compositions to be applied after a procedure chosen from dyeing, bleaching, permanent-waving and straightening the hair; and rinse-out compositions to be applied between the two steps of a procedure chosen from permanent-waving and straightening the hair.

The compositions according to the invention can also be in a form chosen from aqueous and aqueous-alcoholic lotions for a care chosen from skin care and hair care.

The cosmetic compositions according to the invention can be in a form chosen from gels, milks, creams, emulsions, thickened lotions and mousses and can be used for treating at least one keratin material chosen from skin, nails, eyelashes, lips, and hair.

The compositions can be packaged in various forms chosen from vaporizers, pump-dispenser bottles and aerosol containers in order to ensure application of the composition in vaporized form or in the form of a mousse. Such packaging forms are indicated, for example, when it is desired to obtain a spray, a lacquer or a mousse for treating the hair.

In all of the text hereinabove and hereinbelow, the percentages expressed are on a weight basis.

The invention will now be illustrated more fully with-the aid of the examples which follow, which should not be considered as limiting it to the embodiments described. In the examples, AM means active material.

### EXAMPLE 1

A conditioner in accordance with the invention, having the following composition, was prepared:

| | | |
|---|---|---|
| Mixture of myristyl, cetyl and stearyl myristate, | 0.5 | g |
| palmitate and stearate | | |
| Behenyltrimethylammonium chloride (Genamin KDMP | 1.1 | gAM |
| from Clariant) | | |
| Cationic emulsion containing 67% AM of copolymer | 1.36 | gAM |
| of polydimethylsiloxane containing α,ω- | | |
| vinyl groups/polydimethyl-siloxane containing | | |
| α,ω-hydrogeno groups (DC-1997 from Dow | | |
| Corning) | | |
| Mixture of cetyl alcohol and of stearyl alcohol | 3 | g |
| (50/50 by weight) | | |
| Lauryldimethicone copolyol containing 91% AM | 0.23 | gAM |
| (Q2-5200 from Dow Corning) | | |
| Xanthan gum | 0.2 | g |
| Fragrance, preserving agents | | qs |
| Water | qs 100 | g |

This composition is applied to washed and dried hair. It is left to stand on the hair for 2 minutes and is then rinsed off thoroughly with water.

Hair treated with this conditioner is soft, smooth and disentangles easily.

What is claimed is:

1. A cosmetic composition comprising, in a cosmetically acceptable medium, at least one non-cellulose thickener and at least one aqueous emulsion comprising at least one silicone copolymer with a dynamic viscosity ranging from 1×10⁶ to 100×10⁶ cP, resulting from the addition reaction, in the presence of a catalyst, of at least:
(a) one polysiloxane of formula (I): 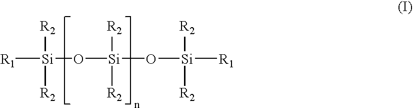
in which:
R₁, which may be identical or different, are independently chosen from groups that can react by chain addition reaction,
R₂ in formula (I), which may be identical or different, are independently chosen from alkyl, alkenyl, cycloalkyl, aryl, hydroxyl, and alkylaryl groups, and can optionally further comprise functional groups,
n is an integer wherein the polysiloxane of formula (I) has a kinematic viscosity ranging from 1 to 1×10⁶ mm²/s; and
(b) at least one silicone compound comprising at least one and not more than two groups capable of reacting with the groups R₁ of the polysiloxane (a), wherein:
at least one of the compounds of type (a) and (b) comprises an aliphatic group comprising an ethylenic unsaturation.

2. A composition according to claim 1, wherein R₁ is chosen from a hydrogen atom and aliphatic groups comprising an ethylenic unsaturation.

3. A composition according to claim 2, wherein the aliphatic groups comprising an ethylenic unsaturation are chosen from vinyl, allyl and hexenyl groups.

4. A composition according to claim 1, wherein the groups R₂ are chosen from hydroxyl groups; alkyl groups comprising from 1 to 20 carbon atoms; cycloalkyl groups comprising from 5 to 6 carbon atoms; phenyl groups; alkylaryl groups comprising from 7 to 20 carbon atoms; and can optionally further comprise functional groups chosen from ethers, amines, carboxyls, hydroxyls, thiols, esters, sulfonates and sulfates.

5. A composition according to claim 1, wherein said alkenyl groups are chosen from alkenyl groups comprising from 2 to 10 carbon atoms.

6. A composition according to claim 1, wherein R₂ is a methyl group.

7. A composition according to claim 1, wherein n is an integer ranging from 5 to 5,000.

8. A composition according to claim 1, wherein the compound of type (b) is another polysiloxane of type (a) in which at least one and not more than two groups R₁ of the polysiloxane (b) can react with the groups R₁ of the polysiloxane (a).

9. A composition according to claim 1, wherein, in the presence of a hydrosilylation catalyst, the at least one silicone copolymer is obtained by addition reaction of at least:
(a) one α,ω-divinylpolydimethylsiloxane, and
(b) one α,ω-dihydrogenopolydimethylsiloxane.

10. A composition according to claim 9, wherein the hydrosilylation catalyst is a platinum catalyst.

11. A composition according to claim 1, wherein said aqueous emulsion of the at least one silicone copolymer has a silicone droplet or particle size ranging from 10 nm to 50 μm.

12. A composition according to claim 11, wherein said emulsion of the at least one silicone copolymer has a silicone droplet or particle size ranging from 0.3 μm to 20 μm.

13. A composition according to claim 1, wherein said aqueous emulsion of the at least one silicone copolymer represents from 0.5% to 15% by weight relative to the total weight of the composition.

14. A composition according to claim 1, wherein the at least one silicone copolymer is essentially non-crosslinked.

15. A composition according to claim 1, wherein the at least one silicone copolymer is present in an amount ranging from 0.05% to 10% by weight relative to the total weight of the composition.

16. A composition according to claim 15, wherein the at least one silicone copolymer is present in an amount ranging from 0.1% to 5% by weight relative to the total weight of the composition.

17. A composition according to claim 1, wherein said at least one non-cellulose thickener is chosen from natural thickeners and synthetic origin thickeners.

18. A composition according to claim 17, wherein said at least one non-cellulose thickener of natural origin is optionally chemically-modified and is chosen from xanthan gum, scleroglucan gum, gellan gum, rhamsan gum, alginates, maltodextrin, starch, starch derivatives, karaya gum, carob flour, and guar gums.

19. A composition according to claim 17, wherein said at least one non-cellulose thickener of synthetic origin is chosen from acrylic acid polymers, methacrylic acid polymers and copolymers obtained from at least one monomer chosen from acrylic acid and methacrylic acid.

20. A composition according to claim 19, wherein said at least one non-cellulose thickener of synthetic origin is chosen from acrylic acid/ethyl acrylate copolymers.

21. A composition according to claim 17, wherein said at least one non-cellulose thickener of synthetic origin is chosen from carboxyvinyl polymers.

22. A composition according to claim 19, wherein said at least one non-cellulose thickener of synthetic origin is chosen from copolymers of acrylamide and an acrylamide derivative.

23. A composition according to claim 1, wherein said at least one non-cellulose thickener is chosen from:
crosslinked 2-acrylamido-2-methylpropanesulfonic homopolymers,
optionally crosslinked copolymers of acrylamide and of ammonium acrylate,
optionally crosslinked copolymers of acrylamide and of methacrylamide and of methacryloyloxyethyltrimethylammonium chloride, and
optionally crosslinked, partially and totally neutralized copolymers of acrylamide and of 2-acrylamido-2-methylpropanesulfonic acid.

24. A composition according to claim 17, wherein said at least one non-cellulose thickener of synthetic origin is chosen from polyethylene glycols and polyethylene glycol derivatives.

25. A composition according to claim 23, wherein said optionally crosslinked copolymers of acrylamide and of ammonium acrylate are chosen from acrylamide/ammonium acrylate copolymers crosslinked with a crosslinking agent comprising polyolefinic unsaturation.

26. A composition according to claim 25, wherein said optionally crosslinked copolymers of acrylamide and of ammonium acrylate are chosen from acrylamide/ammonium acrylate copolymers (5/95 by weight) crosslinked with a crosslinking agent comprising polyolefinic unsaturation.

27. A composition according to claim 25, wherein said crosslinking agent is chosen from divinylbenzene, tetraallyloxyethane, methylenebisacrylamide, diallyl ether, polyallylpolyglyceryl ethers and allyl ethers of alcohols of the sugar series.

28. A composition according to claim 27, wherein said allyl ethers of alcohols of the sugar series are chosen from erythritol, pentaerythritol, arabitol, mannitol, sorbitol and glucose.

29. A composition according to claim 23, wherein said copolymers of acrylamide and of 2-acrylamido-2-methylpropanesulfonic acid are chosen from copolymers crosslinked with a compound comprising polyolefinic unsaturation, and partially or totally neutralized with a neutralizing agent.

30. A composition according to claim 29, wherein said neutralizing agent is chosen from sodium hydroxide, potassium hydroxide, aqueous ammonia and amines.

31. A composition according to claim 30, wherein said amines are chosen from triethanolamine and monoethanolamine.

32. A composition according to claim 23, wherein said crosslinked copolymers of acrylamide and of methacryloyloxyethyltrimethylammonium chloride are chosen from copolymers obtained by copolymerization of acrylamide and of dimethylaminoethyl methacrylate quaternized with methyl chloride, followed by crosslinking with a compound comprising olefinic unsaturation.

33. A composition according to claim 32, wherein said compound comprising olefinic unsaturation is chosen from methylenebisacrylamide.

34. A composition according to claim 17, wherein said at least one non-cellulose thickener is present in an amount ranging frm 0.001% to 20% by weight relative to the total weight of the composition.

35. A composition according to claim 34, wherein said at least one non-cellulose thickener is present in an amount ranging from 0.01% to 10% by weight relative to the total weight of the composition.

36. A composition according to claim 35, wherein said at least one non-cellulose thickener is present in an amount ranging from 0.1% to 3% by weight relative to the total weight of the composition.

37. A composition according to claim 1 further comprising at least one cationic surfactant chosen from:
A) quaternary ammonium salts of formula (IV) below: 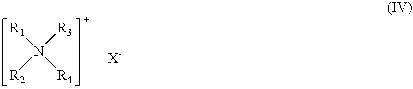
in which:
the radicals R₁, R₂, R₃, and R₄, which may be identical or different, are independently chosen from linear and branched aliphatic radicals comprising from 1 to 30 carbon atoms, and aromatic radicals, wherein the aliphatic radicals optionally comprise hetero atoms, and
X⁻ is an anion chosen from the group of halides, phosphates, anions derived from organic acids, (C₂-C₆)alkyl sulfates, alkyl sulfonates, and alkylaryl sulfonates;
B) quaternary ammonium salts of imidazolinium of formula (V) below: 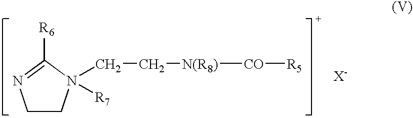
in which:
R₅ is chosen from alkenyl and alkyl radicals comprising from 8 to 30 carbon atoms,
R₆ is chosen from a hydrogen atom, C₁-C₄ alkyl radicals, and alkenyl and alkyl radicals comprising from 8 to 30 carbon atoms,
R₇ is chosen from C₁-C₄ alkyl radicals,
R₈ is chosen from a hydrogen atom and C₁-C₄ alkyl radicals, and
X⁻ is an anion chosen from halides, phosphates, acetates, lactates, alkyl sulfates, alkyl sulfonates, and alkylaryl sulfonates;
C) diquaternary ammonium salts of formula (VI): 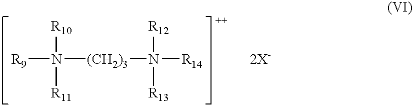
in which:
R₉ is chosen from aliphatic radicals comprising from 16 to 30 carbon atoms,
R₁₀, R₁₁, R₁₂, R₁₃ and R₁₄, which may be identical or different, are independently chosen from a hydrogen atom and alkyl radicals comprising from 1 to 4 carbon atoms, and
X⁻ is an anion chosen from halides, acetates, phosphates, nitrates and methyl sulfates;
D) quaternary ammonium salts of formula (VII) below comprising at least one ester function: 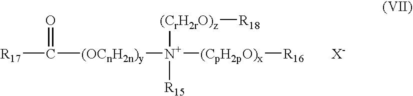
in which:
R₁₅ is chosen from C₁-C₆ alkyl radicals and C₁-C₆ hydroxyalkyl and C₁-C₆ dihydroxyalkyl radicals;
R₁₆ is chosen from:
acyl groups of the following formula: 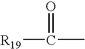
wherein R₁₉ is defined below,
linear and branched, saturated and unsaturated, C₁-C₂₂ hydrocarbon-based radicals, and
a hydrogen atom;
R₁₈ is chosen from:
acyl groups of the following formula: 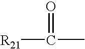
wherein R₂₁ is defined below,
linear and branched, saturated and unsaturated, C₁-C₆ hydrocarbon-based radicals, and
a hydrogen atom;
R₁₇, R₁₉ and R₂₁, which may be identical or different, are independently chosen from linear and branched, saturated and unsaturated, C₇-C₂₁ hydrocarbon-based radicals;
n, p and r, which may be identical or different, are independently integers ranging from 2 to 6;
y is an integer ranging from 1 to 10;
x and z, which may be identical or different, are independently integers ranging from 0 to 10; and
X⁻ is chosen from simple and complex, organic and inorganic anions; and
provided that the sum x+y+z is from 1 to 15, and that when x is 0, then R₁₆ is chosen from linear and branched, saturated and unsaturated, C₁-C₂₂ hydrocarbon-based radicals, and that when z is 0, then R₁₈ is chosen from linear and branched, saturated and unsaturated, C₁-C₆ hydrocarbon-based radicals.

38. A composition according to claim 37, wherein said at least one cationic surfactant is chosen from:
A) quaternary ammonium salts of formula (IV) below: 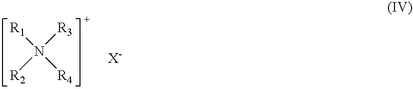
wherein:
X⁻ is an anion chosen from halides, (C₂-C₆)alkyl sulfates, phosphates, alkyl and alkylaryl sulfonates, and anions derived from organic acids, and
i) the radicals R₁, R₂, and R₃, which may be identical or different, are independently chosen from linear and branched aliphatic radicals comprising from 1 to 4 carbon atoms, optionally comprising hetero atoms, and aromatic radicals, and
R₄ is chosen from linear and branched alkyl radicals comprising from 16 to 30 carbon atoms;
ii) the radicals R₁ and R₂, which may be identical or different, are independently chosen from linear and branched aliphatic radicals comprising from 1 to 4 carbon atoms, optionally comprising hetero atoms, and aromatic radicals, and
R₃ and R₄, which may be identical or different, are independently chosen from linear and branched alkyl radicals comprising from 12 to 30 carbon atoms, wherein said radicals further comprise at least one function chosen from ester and amide functions.

39. A composition according to claim 37, wherein in said quaternary ammonium salts of formula (VII):
R₁₅ is chosen from methyl and ethyl radicals,
x and y are equal to 1;
z is equal to 0 or 1;
n, p and r are equal to 2;
R₁₆ is chosen from:
acyl radicals 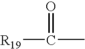
wherein R₁₉ is defined below,
methyl, ethyl and C₁₄-C₂₂ hydrocarbon-based radicals, and
a hydrogen atom;
R₁₈ is chosen from:
acyl radicals 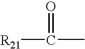
wherein R₂₁ is defined below,
a hydrogen atom; and
R₁₇, R₁₉ and R₂₁, which may be identical or different, are independently chosen from linear and branched, saturated and unsaturated, C₁₃-C₁₇ hydrocarbon-based radicals.

40. A composition according to claim 39, wherein R₁₇, R₁₉ and R₂₁ are chosen from linear and branched, saturated and unsaturated C₁₃-C ₁₇ aliphatic radicals.

41. A composition according to claim 39, wherein the hydrocarbon-based radicals are chosen from linear hydrocarbon-based radicals.

42. A composition according to claim 37, wherein the compounds of formula (VII) are chosen from diacyloxyethyldimethylammonium, diacyloxyethylhydroxyethylmethylammonium, monoacyloxyethyldihydroxyethylmethylammonium, tracyloxyethylmethylammonium and monoacyloxyethylhydroxyethyldimethylammonium salts.

43. A composition according to claim 42, wherein said monoacyloxyethyl-hydroxyethyidimethylammonium salts are chosen from monoacyloxyethyl hydroxyethyldimethylammonium chloride salts and monoacyloxyethyl hydroxyethyldimethylammonium methyl sulfate salts.

44. A composition according to claim 39, wherein when R₁₆ and R₁₈ are chosen from acyl radicals, said acyl radicals are obtained from plant oils chosen from palm oil and sunflower oil.

45. A composition according to claim 37, wherein X⁻ of said quaternary ammonium salts of formula (IV) is an anion chosen from chloride, bromide, iodide, methyl sulfate, acetate, and lactate.

46. A composition according to claim 37, wherein said aromatic radicals of said quaternary ammonium salts of formula (IV) are chosen from aryl and alkylaryl.

47. A composition according to claim 37, wherein said hetero atoms of said quaternary ammonium salts of formula (IV) are chosen from oxygen, nitrogen, sulfur and halogens.

48. A composition according to claim 38, wherein said aliphatic radicals of formula (IV)(ii) are chosen from alkyl, alkoxy, alkylamide, polyoxy(C₂-C₆)alkylene, and hydroxyalkyl radicals comprising from 1 to 4 carbon atoms.

49. A composition according to claim 38, wherein said R₃ and R₄ of formula (IV)(ii) are chosen from (C₁₂-C₂₂)alkylamido(C₂-C₆)alkyl and (C₁₂-C₂₂)alkylacetate radicals.

50. A composition according to claim 37, wherein said R₅ of formula (V) is chosen from radicals derived from tallow fatty acid.

51. A composition according to claim 37, wherein in said quaternary ammonium salts of imidazolinium of formula (V):
R₅ and R₆, which may be identical or different, are independently chosen from alkenyl and alkyl radicals comprising from 12 to 21 carbon atoms,
R₇ is methyl, and
R₈ is hydrogen.

52. A composition according to claim 51, wherein said R₅ and R₆, which may be identical or different, are independently chosen from radicals derived from tallow fatty acid.

53. A composition according to claim 37, wherein said diquaternary ammonium salts comprise propane tallow diammonium dichloride.

54. A composition according to claim 37, wherein said R₁₅ alkyl radicals of said quaternary ammonium salts of formula (VII) are chosen from linear and branched C₁-C₆ alkyl radicals.

55. A composition according to claim 54, wherein said R₁₅ radicals are linear radicals.

56. A composition according to claim 55, wherein said R₁₆ radicals are chosen from methyl, ethyl, hydroxyethyl and dihydroxypropyl.

57. A composition according to claim 56, wherein said R₁₅ radicals are chosen from methyl and ethyl.

58. A composition according to claim 37, wherein said sum of x+y+z of said quaternary ammonium salts of formula (VII) ranges from 1-10.

59. A composition according to claim 37, wherein said quaternary ammonium salts of formula (IV) are chosen from (a) compounds comprising at least two fatty aliphatic radicals comprising from 8 to 30 carbon atoms, (b) compounds comprising at least one fatty aliphatic radical comprising from 17 to 30 carbon atoms, and (c) compounds comprising at least one aromatic radical.

60. A composition according to claim 37, wherein said at least one cationic surfactant is chosen from behenyltrimethylammonium salts, stearamidopropyldimethyl(myristyl acetate)ammonium salts, Quaternium-27 and Quaternium-83.

61. A composition according to claim 37, wherein the at least one cationic surfactant is present in an amount ranging from 0.1% to 10% by weight relative to the total weight of the composition.

62. A composition according to claim 61, wherein the at least one cationic surfactant is present in an amount ranging from 0.5% to 7% by weight relative to the total weight of the composition.

63. A composition according to claim 62, wherein the at least one cationic surfactant is present in an amount ranging from 1% to 5% by weight relative to the total weight of the composition.

64. A composition according to claim 1 further comprising at least one surfactant chosen from anionic, nonionic, and amphoteric surfactants.

65. A composition according to claim 64, wherein the at least one surfactant chosen from anionic, nonionic, and amphoteric surfactants is present in an amount ranging from 0.1% to 60% by weight, relative to the total weight of the composition.

66. A composition according to claim 65, wherein the at least one surfactant chosen from anionic, nonionic, and amphoteric surfactants is present in an amount ranging from 3% to 40% by weight, relative to the total weight of the composition.

67. A composition according to claim 66, wherein the at least one surfactant chosen from anionic, nonionic, and amphoteric surfactants is present in an amount ranging from 5% to 30% by weight, relative to the total weight of the composition.

68. A composition according to claim 64, wherein the at least one surfactant chosen from anionic, nonionic, and amphoteric surfactants comprises at least one anionic surfactant salt chosen from alkyl sulfates, alkyl ether sulfates, alkylamidoether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates.

69. A composition according to claim 64, wherein said at least one surfactant is chosen from anionic surfactants chosen from alkaline salts, sodium salts, ammonium salts, amine salts, amino alcohol salts and magnesium salts.

70. A composition according to claim 68, wherein said alkyl and acyl portions of radicals of said salts comprise 1 and from 8 to 24 carbon atoms, and said aryl portions of radicals of said salts are phenyl.

71. A composition according to claim 64, wherein said at least one surfactant is chosen from anionic surfactants chosen from fatty acid salts, acyl lactates wherein the acyl radical comprises 8 to 20 carbon atoms, and weakly anionic surfactants.

72. A composition according to claim 71, wherein said fatty acid salts are chosen from the salts of oleic acid, ricinoleic acid, palmitic acid, stearic acid, coconut oil acid and hydrogenated coconut oil acid.

73. A composition according to claim 64, wherein said at least one surfactant is chosen from anionic surfactants chosen from alkyl-D-galactosiduronic acids and their salts, polyoxyalkylenated (C₆-C₂₄)alkyl ether carboxylic acids and their salts, polyoxyalkylenated (C₆-C₂₄)alkylaryl ether carboxylic acids and their salts, and polyoxyalkylenated (C₆-C₂₄)alkylamido ether carboxylic acids and their salts.

74. A composition according to claim 71, wherein said weakly anionic surfactants comprise from 2 to 50 ethylene oxide groups.

75. A composition according to claim 68, wherein said at least one anionic surfactant salt is chosen from alkyl sulfates and alkyl ether sulfates.

76. A composition according to claim 64, wherein said at least one surfactant is chosen from nonionic surfactants chosen from, polyethoxylated, polypropoxylated and polyglycerolated fatty acids, alkylphenols, α-diols and alcohols having a fatty aliphatic chain comprising 8 to 18 carbon atoms, wherein the number of ethylene oxide and propylene oxide groups ranges from 2 to 50 and the number of glycerol groups ranges from 2 to 30, copolymers of ethylene oxide and of propylene oxide, condensates of ethylene oxide and of propylene oxide with fatty alcohols, polyethoxylated fatty amides comprising from 2 to 30 mol of ethylene oxide, polyglycerolated fatty amides comprising on average 1 to 5 glycerol groups, polyethoxylated fatty amines comprising from 2 to 30 mol of ethylene oxide, oxyethylenated fatty acid esters of sorbitan comprising from 2 to 30 mol of ethylene oxide, fatty acid esters of sucrose, fatty acid esters of polyethylene glycol, alkylpolyglycosides, N-alkylglucamine derivatives, and amine oxides.

77. A composition according to claim 76, wherein said polyglycerolated fatty amides comprise on average 1.5 to 4 glycerol groups.

78. A composition according to claim 76, wherein said amine oxides are chosen from (C₁₀-C₁₄)alkylamine oxides and N-acylaminopropylmorpholine oxides.

79. A composition according to claim 76, wherein said nonionic surfactants are chosen from alkylpolyglycosides.

80. A composition according to claim 64, wherein said at least one surfactant is chosen from amphoteric surfactants chosen from aliphatic secondary and tertiary amine derivatives wherein the aliphatic radical is chosen from linear and branched chain radicals comprising 8 to 22 carbon atoms and comprising at least one water-soluble anionic group, (C₈-C₂₀)alkylbetaines, sulfobetaines, (C₈-C₂₀)alkylamido(C₁-C₆)alkylbetaines, and (C₈-C₂₀)alkylamido(C₁-C₆)alkylsulfobetaines.

81. A composition according to claim 80, wherein said at least one water-soluble anionic group is chosen from carboxylates, sulfonates, sulfates, phosphates and phosphonates.

82. A composition according to claim 80, wherein said amine derivatives are chosen from the compounds:
R₂—CONHCH₂CH₂—N⁺(R₃)(R₄)(CH₂COO—) (2)
in which:
R₂ is chosen from alkyl radicals derived from an acid R₂—COOH present in hydrolysed coconut oil, heptyl, nonyl and undecyl radicals,
R₃ is chosen from β-hydroxyethyl groups, and
R₄ is chosen from carboxymethyl groups;
and
R₅—CONHCH₂CH₂—N(B)(C) (3)
in which:
(B) is —CH₂CH₂OX′, with X′ chosen from a —CH₂CH₂—COOH group and a hydrogen atom,
(C) is —(CH₂)_{z}—Y′, with z=1 or 2, and with Y⁻ chosen from —COOH and —CH₂—CHOH—SO₃H radicals,
R₅ is chosen from alkyl radicals and unsaturated C₁₇ radicals.

83. A composition according to claim 82, wherein said alkyl radicals R₅ are chosen from (a) alkyl radicals of an acid R₅—COOH present in oils chosen from coconut oil and hydrolysed linseed oil, and (b) C₁₇ alkyl radicals and the iso forms.

84. A composition according to claim 82, wherein said alkyl radicals of said R₅ are chosen from alkyl radicals chosen from C₇, C₉, C₁₁ and C₁₃ alkyl radicals.

85. A composition according to claim 64, wherein said at least one surfactant is chosen from at least two surfactants of different types.

86. A composition according to claim 85, wherein said at least two surfactants of different types are chosen from (a) more than one anionic surfactant, (b) at least one anionic surfactant and at least one amphoteric surfactant, and (c) at least one anionic surfactant and and at least one nonionic surfactant.

87. A composition according to claim 64, wherein said at least one surfactant is chosen from anionic surfactants chosen from (C₁₂-C₁₄)alkyl sulfates of sodium, of triethanolamine and of ammonium, (C₁₂-C₁₄)alkyl ether sulfates of sodium, of triethanolamine and of ammonium, oxyethylenated with 2.2 mol of ethylene oxide, sodium cocoyl isethionate, and sodium (C₁₄-C₁₆)-α-olefin sulfonate, and from an amphoteric surfactant chosen from either:
amine derivatives comprising disodium cocoamphodipropionate and sodium cocoamphopropionate, or
amphbteric surfactants of zwitterionic type.

88. A composition according to claim 87, wherein said amphoteric surfactants of zwitterionic type are chosen from alkylbetaines.

89. A composition according to claim 88, wherein said alkylbetaines are chosen from cocobetaine.

90. A composition according to claim 1 further comprising at least one additive chosen from fragrances, nacreous agents, preserving agents, silicone sunscreens, non-silicone sunscreens, vitamins, provitamins, cationic, amphoteric, anionic and nonionic polymers, proteins, protein hydrolysates, 18-methyleicosanoic acid, hydroxy acids, panthenol, volatile and non-volatile, cyclic and linear and crosslinked, modified and non-modified silicones, ceramides, pseudoceramides, and plant, animal, mineral and synthetic oils.

91. A composition according to claim 90, wherein said at least one additive is present in an amount ranging from 0 to 20% by weight relative to the total weight of the composition.

92. A rinse-out conditioner, a leave-in conditioner, a composition for permanent-waving the hair, a composition for straightening the hair, a composition for dyeing the hair, a composition for bleaching the hair, a rinse-out composition to be applied before a procedure chosen-from dyeing, bleaching, permanent-waving and straightening the hair, a rinse-out composition to be applied after a procedure chosen from dyeing, bleaching, permanent-waving and straightening the hair, a rinse-out composition to be applied between the two steps of a permanent-waving operation, a rinse-out composition to be applied between the two steps of a hair-straightening operation, a washing composition for the body, an aqueous lotion, an aqueous-alcoholic lotion, a gel, a milk, a cream, an emulsion, a thickened lotion, a mousse, or a detergent composition comprising a washing base comprising, in a cosmetically acceptable medium, at least one non-cellulose thickener and at least one aqueous emulsion comprising at least one silicone copolymer with a dynamic viscosity ranging from 1×10⁶ to 100×10⁶ cP, resulting from the addition reaction, in the presence of a catalyst, of at least:
(a) one polysiloxane of formula (I): 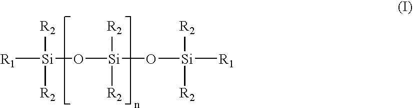
in which:
R₁, which may be identical or different, are independently chosen from groups that can react by chain addition reaction,
R₂ in formula (I), which may be identical or different, are independently chosen from alkyl, alkenyl, cycloalkyl, aryl, hydroxyl, and alkylaryl groups, and can optionally further comprise functional groups,
n is an integer wherein the polysiloxane of formula (I) has a kinematic viscosity ranging from 1 to 1×10⁶ mm²/s; and
(b) at least one silicone compound comprising at least one and not more than two groups capable of reacting with the groups R₁ of the polysiloxane (a), wherein:
at least one of the compounds of type (a) and (b) comprises an aliphatic group comprising an ethylenic unsaturation.

93. An aqueous or aqueous-a1coholic lotion according to claim 92, said lotion being suitable for skin care or for hair care.

94. A gel, a milk, a cream, an emulsion, a thickened lotion or a mousse according to claim 92, wherein said gel, milk, cream, emulsion, thickened lotion or mousse is suitable to be applied to at least one keratin material chosen from skin, nails, eyelashes, lips and hair.

95. A detergent composition comprising a washing base according to claim 92, wherein said composition is chosen from shampoos, shower gels, bubble baths and make-up-removing products.

96. A detergent composition comprising a washing base according to claim 92, wherein said washing base comprises at least one surfactant chosen from anionic, amphoteric, nonionic and cationic surfactants.

97. A detergent composition according to claim 96, wherein said at least one surfactant is present in an amount effective to provide satisfactory foaming power and satisfactory detergent power.

98. A detergent composition comprising a washing base according to claim 96, wherein said washing base is present in an amount ranging from 4% to 50% by weight, relative to the total weight of the final composition.

99. A detergent composition comprising a washing base according to claim 98, wherein said washing base is present in an amount ranging from 6% to 35% by weight, relative to the total weight of the final composition.

100. A detergent composition comprising a washing base according to claim 99, wherein said washing base is present in an amount ranging from 8% to 25% by weight, relative to the total weight of the final composition.

101. A process of washing or caring for a keratin material comprising applying to said keratin material a composition comprising, in a cosmetically acceptable medium, at least one non-cellulose thickener and at least one aqueous emulsion comprising at least one silicone copolymer with a dynamic viscosity ranging from 1×10⁶ to 100×10⁶ cP, resulting from the addition reaction, in the presence of a catalyst, of at least:
(a) one polysiloxane of formula (I): 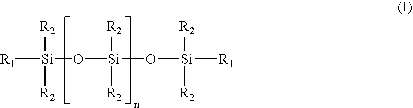
in which:
R₁, which may be identical or different, are independently chosen from groups that can react by chain addition reaction,
R₂ in formula (I), which may be identical or different, are independently chosen from alkyl, alkenyl, cycloalkyl, aryl, hydroxyl, and alkylaryl groups, and can optionally further comprise functional groups,
n is an integer wherein the polysiloxane of formula (I) has a kinematic viscosity ranging from 1 to 1×10⁶ mm²/s; and
(b) at least one silicone compound comprising at least one and not more than two groups capable of reacting with the groups R₁ of the polysiloxane (a), wherein:
at least one of the compounds of type (a) and (b) comprises an aliphatic group comprising an ethylenic unsaturation.

102. A process for treating a keratin material comprising applying to said keratin material a composition comprising, in a cosmetically acceptable medium, at least one non-cellulose thickener and at least one aqueous emulsion comprising at least one silicone copolymer with a dynamic viscosity ranging from 1×10⁶ to 100×10⁶ cP, resulting from the addition reaction, in the presence of a catalyst, of at least:
(a) one polysiloxane of formula (I): 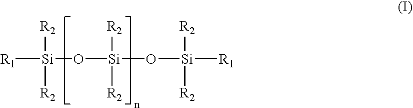
in which:
R₁, which may be identical or different, are independently chosen from groups that can react by chain addition reaction,
R₂ in formula (I), which may be identical or different, are independently chosen from alkyl, alkenyl, cycloalkyl, aryl, hydroxyl, and alkylaryl groups, and can optionally further comprise functional groups,
n is an integer wherein the polysiloxane of formula (I) has a kinematic viscosity ranging from 1 to 1×10⁶ mm²/s; and
(b) at least one silicone compound comprising at least one and not more than two groups capable of reacting with the groups R₁ of the polysiloxane (a), wherein:
at least one of the compounds of type (a) and (b) comprises an aliphatic group comprising an ethylenic unsaturation,
and optionally rinsing said composition out with water.

103. A process for washing or treating a keratin material according to claim 102, wherein said keratin material is chosen from hair, skin, eyelashes, eyebrows, nails, lips and scalp.

104. A process for manufacturing a cosmetic product comprising including in said product at least one non-cellulose thickener and at least one aqueous emulsion comprising at least one silicone copolymer with a dynamic viscosity ranging from 1×10⁶ to 100×10⁶ cP, resulting from the addition reaction, in the presence of a catalyst, of at least:
(a) one polysiloxane of formula (I): 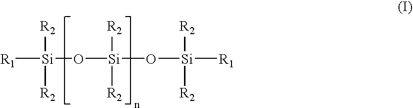
in which:
R₁, which may be identical or different, are independently chosen from groups that can react by chain addition reaction,
R₂ in formula (I), which may be identical or different, are independently chosen from alkyl, alkenyl, cycloalkyl, aryl, hydroxyl, and alkylaryl groups, and can optionally further comprise functional groups,
n is an integer wherein the polysiloxane of formula (I) has a kinematic viscosity ranging from 1 to 1×10⁶ mm²/s; and
(b) at least one silicone compound comprising at least one and not more than two groups capable of reacting with the groups R₁ of the polysiloxane (a), wherein:
at least one of the compounds of type (a) and (b) comprises an aliphatic group comprising an ethylenic unsaturation.
